(19) United States
(12) Patent Application Publication
(10) Pub. No.: US 2014/0141459 A1
(43) Pub. Date: May 22, 2014

(54) PENTRAXIN 3 ASSOCIATION WITH ASTHMA

(71) Applicants: Rachel Bunting, Pottstown, PA (US); Cory M. Hogaboam, Santa Monica, CA (US); Ken Kilgore, Spring House, PA (US)

(72) Inventors: Rachel Bunting, Pottstown, PA (US); Cory M. Hogaboam, Santa Monica, CA (US); Ken Kilgore, Spring House, PA (US)

(73) Assignees: THE REGENTS OF THE UNIVERSITY OF MICHIGAN, Ann Arbor, MI (US); JANSSEN BIOTECH, INC., Horsham, PA (US)

(21) Appl. No.: 14/235,111

(22) PCT Filed: Jul. 27, 2012
(86) PCT No.: PCT/US12/48482

§ 371 Date: Jan. 27, 2014

Related U.S. Application Data
(60) Provisional application No. 61/512,482, filed on Jul. 28, 2011.

(51) Int. Cl. G01N 33/68

(52) U.S. Cl. CPC G01N 33/6893
USPC 435/7.94; 435/7.92

(57) ABSTRACT

The present invention relates to methods of utilizing pentraxin 3 as a marker for therapeutic efficacy in preclinical models of asthma.

This application claims priority to U.S. No. 61/512,482, filed Jul. 28, 2012, which is entirely incorporated herein by reference.

### BACKGROUND OF THE INVENTION

1. Field of the Invention

The present invention relates to methods of utilizing pentraxin 3 as a marker for therapeutic efficacy in preclinical models of asthma.

2. Background of the Invention

Asthma, a chronic inflammatory condition of the airways in the lung has become a major public health concern in recent years. Currently, an estimated 300 million people worldwide have been diagnosed with asthma and the number is expected to increase by 33% to 400 million by 2025 (Masoli et al., Allergy 59:469-478, 2004; Beasley et al., J Allergy Clin Immunol 105:5466-5472, 2000). The pathogenesis of asthma is associated with several molecular and cellular pathways, including allergic, non-allergic and intrinsic pathways. Asthma is characterized by airway hyperresponsiveness (AHR), bronchoconstriction, wheezing, eosinophilic or neutrophilic inflammation, mucus hypersecretion, goblet cell metaplasia, or airway wall remodeling events, escpecially subepithelial fibrosis and smooth muscle cell hyperplasia (Kim et al., Nature Immunol 11:577-584; Shalaby and Martin, Current Opin Pharamc 10:218-225, 2010). Patients with asthma experience exacerbations, a worsening of symptoms, most commonly due to microbial infections of the respiratory tract (e.g. rhinovirus, influenza virus, Haemophilus influenza, etc.). Asthmatic attacks can be triggered by environmental factors (e.g. ascarids, insects, animals (e.g., cats, dogs, rabbits, mice, rats, hamsters, guinea pigs and birds), fungi, air pollutants (e.g., tobacco smoke), irritant gases, fumes, vapors, aerosols, chemicals, pollen, exercise, or cold air. Apart from asthma, several chronic inflammatory diseases affecting the lung are characterized by neutrophil infiltration to the airways and/or airway hyperresponsiveness, for example chronic obstructive pulmonary disease (COPD), bacterial pneumonia, cystic fibrosis, and allergic rhinitis (Linden et al., Eur Respir J 15:973-977, 2000; Rahman et al., Clin Immunol 115:268-276, 2005; Fahy and O'Byrne, Am J Respir Crit. Care Med 163:822-823, 2001).

Preclinical in vivo models recapitulating many features of asthma have been developed. For example, ovalbumin or clinically relevant allergens such as house dust mice or fungal allergens are used to induce inflammatory changes and airway remodeling that results from allergen exposures. Models of viral exacerbations of asthma and steroid-resistant asthma have been developed (for review, see Stevenson and Birrell, Pharamcol Ther Nov. 11, 2010 [epub ahead of print]).

Evaluation of changes at the protein and gene expression levels in the lungs of preclinical in vivo models can provide information of molecular mechanisms of asthma, identify possible biomarkers for the disease in humans, and identify possible disease-causative proteins. Further, biomarkers correlating with various asthma phenotypes are needed in assessing efficacy of potential therapeutics.

### BRIEF DESCRIPTION OF THE DRAWINGS

FIG. 1. Bronchoalveolar larvage (BAL) cells in a time course of an A) ovalbumin (OVA) induced model of asthma and B) in control mice administered PBS. Lymph=lymphocytes, Monos=monocytes, PMN=peripheral mononuclear cells, Eos=eosinophiles.

FIG. 2. Time course of IL-5, IL-4, and IL-6 protein levels in lung homogenates (LH) in OVA induced model of asthma.

FIG. 3. Time course of OVA-specific IgE levels in serum in OVA-induced model of asthma.

FIG. 4. Increased pentraxin 3 (PTX3) levels in lung homogenates at indicated days in an OVA-induced model of asthma.

FIG. 5. BAL cells in A) house dust mite (HDM) induced model of asthma and B) in control mice administered PBS.

FIG. 6. IL-4 and IL-5 levels in lung homogenates in HDM induced model of asthma.

FIG. 7. Increased PTX3 levels in lung homogenates in HDM induced model of asthma.

FIG. 8. PTX3 levels in A) cultured macrophages; B) lung homogenates in A. fumigatus induced model of asthma.

FIG. 9. Dexamethasone (Dex) treatment reduces PTX3 levels in lung homogenates in OVA induced model of asthma; day 25 post-OVA sensitization.

FIG. 10. Increased PTX3 levels in lung biopsies from human asthmatic subjects.

FIG. 11. PTX3 administration induces airway hyper-responsiveness at 15 days but not at 30 days after conidia challenge in A. fumigatus induced model of asthma.

FIG. 12. Levels of indicated cytokines in lung homogenates of PBS or PTX3-treated mice at 30 days after conidia challenge in A. fumigatus induced model of asthma.

### SUMMARY OF THE INVENTION

One aspect of the invention is a method of assessing efficacy of a therapeutic for the treatment of asthma, comprising: providing the therapeutic; providing a first and a second member of a preclinical model of asthma; administering the therapeutic to the first member of the preclinical model of asthma and administering a vehicle to the second member of the preclinical model of asthma; measuring the amount of pentraxin 3 in lung samples of the first member and the second member of the preclinical model of asthma; determining that the lung sample of the first member of the preclinical model of asthma exhibits reduced amount of pentraxin 3 when compared to the amount of pentraxin 3 in the lung sample of the second member of the preclinical model of asthma.

### DETAILED DESCRIPTION OF THE INVENTION

All publications, including but not limited to patents and patent applications, cited in this specification are herein incorporated by reference as though fully set forth.

It is to be understood that the terminology used herein is for the purpose of describing particular embodiments only and is not intended to be limiting. Unless defined otherwise, all technical and scientific terms used herein have the same meaning as commonly understood by one of ordinary skill in the art to which the invention pertains.

Although any methods and materials similar or equivalent to those described herein can be used in the practice for testing of the present invention, exemplary materials and methods are described herein. In describing and claming the present invention, the following terminology will be used.

The term “pentraxin 3” (“PTX3”) refers to a PTX3 polypeptide. The term PTX3 includes human PTX3 and orthologs of the human PTX3, for example PTX3 from primates, rodents, mouse, rat, dogs, and the like. The amino acid sequences of human PTX3 and its orthologs are known and shown in SEQ ID NOs: 1-5 for human, mouse, rat, chimpanzee (Pan Troglodytes), and rhesus monkey (Macaca Mulatta).

The term “asthma” as used herein refers to a chronic condition in a subject, which in most cases is characterized by airway inflammation and airway hypereactivity (AHR), and by symptoms of recurrent coughing, wheezing and shortness of breath. The triggers for asthma include for example an environmental stimulant, such as an allergen (ragweed, house dust, animal hair, pollen, etc.), cold air, warm air, moist air, change in temperature or humidity, upper respiratory infections, exercise, exertion, physical or emotional stress, smoke, viral illnesses such as those caused by common cold. Asthma phenotypes include for example allergic asthma, steroid-resistant asthma, asthma induced by exposure to cigarette smoke or air pollution, exercise, diesel exhaust particles, aspirin, or obesity. The different asthma phenotypes can coexist and act in synergy in patients. “Asthma” includes also asthma exacerbations, induced for example by viruses such as respiratory syncytial virus (RSV).

At the molecular and cellular level, asthma is characterized by at least one “asthma molecular phenotype” present in at least one tissue, cell, or serum sample, for example in lung biopsy, lung homogenate, supernatant of lung homogenate, or serum. Exemplary asthma molecular phenotypes are eosinophilia, neutrophilia, recruitment of Th2 cells and increased Th2 cytokine production, for example increased IL-4, IL-5, or IL-6 production, increased mast cell degranulation, increased IgE levels, increased mucus secretion, goblet cell hyperplasia, increased activation of the innate immune system and pathways, for example NKT cells and macrophages, activation of Th17 cells and increased IL-17 or IL-21 secretion, increased secretion of TSLP, IL-25, or IL-33, and airway remodeling, such as subepithelial fibrosis.

The term “therapeutic” as used herein means a molecule that is believed to provide a therapeutic benefit in an animal patient belonging to any classification. Examples of such animals include mammals such as humans, rodents, dogs, cats and farm animals. The therapeutic may be a small molecule, protein, antibody, antibody fragment, peptide, oligonucleotide, siRNA, and the like. The therapeutic may provide its beneficial effect by any mechanism, for example by reducing inflammation in the lung airways, or by reducing aiwary remodeling.

The term “preclinical model of asthma” as used herein means any animal model exhibiting at least one asthma molecular phenotype.

This invention is based at least in part on the finding that pentraxin 3 (PTX3) levels correlate with at least one asthma molecular phenotype in tissue samples of several preclinical models of asthma, and that administration of standard asthma care medication glucocorticoids to the preclinical models of asthma reduces PTX3 levels. PTX3 thus can serve as a marker for asthma and asthma molecular phenotypes, acute exacerbation in asthma as well as a marker to assess potential efficacy of the therapeutics in preclinical models of asthma.

### Pentraxin 3

PTX3 is a member of a superfamily of acute phase reactants characterized by a cyclic multimeric structure. PTX3 has been shown to play a protective role against certain pathogens such as Aspergillus fumigatus and Influenza viruses (Garlanda et al., Nature 420:182-186, 2002; Reading et al., J Immunol 180:3391-3398, 2008). PTX3 is produced by various cell types, including endothelial cells, monocytes and macrophages (Vouret-Craviari et al., Infect. Immun. 65: 1343-1350, 1997), and mononuclear phagocytes (Vidal Alles et al., Blood 84: 3483-3493, 1994), in response to primary mediators of inflammation such as Interleukin-1β (IL-1β), tumor necrosis factor α (TNF-α) and bacterial products (Basile et al., J. Biol. Chem. 272: 8172-8178, 1997). PTX3 binds to apoptotic cells and debris in response to tissue damage, activating the complement cascade (Garlanda et al., Ann Rev Immunol 23:337-366, 2005).

PTX3 levels have been shown to be increased in diseases associated with tissue injury such as Rheumatoid Arthritis, Atherosclerosis, SLE nephritis, and ventilator-induced lung injury (Luchetti et al., Clin Exp Immunol 119:196-202, 2000; Savchenko et al., J Pathol 215:48-55, 2008; Manfredi et al., Curr Opin Immunol 20:538-544, 2008; Okutani et al., Am J Physiol Lung Cell Mol Physiol 292:L144-153, 2007). PTX3 therapy is suggested for infectious or inflammatory diseases (WO03/011326), autoimmune diseases (WO02/36151), and cancer (WO03/084561; WO03/072603).

### Preclinical Models of Asthma

A spectrum of preclinical models of asthma are well known, each of which recapitulate several asthma molecular phenotypes. Exemplary species that may be used to generate preclinical models of asthma are mice, rats, guinea pigs, sheep, dogs, pigs, and primates. Various asthma models are reviewed in Bates et al., Am J Physiol Lung Cell Mol Physiol 297:L401-L410, 2009; Stevenson and Birrell, Pharmacol Ther 130:93-105, 2011; Van der Velden and Snibson, Pulm Pharmacol Ther Feb. 26, 2011 [Epub ahead of print]; Nials and Uddin, Dis Model Mech 1:213-220, 2008.

An ovalbumin (OVA)-induced asthma model is an exemplary model of allergic asthma. Ovalbumin derived from chicken egg used as an allergen induces a robust, allergic pulmonary inflammation in rodents. Animals are sensitized typically by multiple systemic administrations of the allergen over a period of for example 14-21 days, followed by a challenge with the allergen via the airway over a period of several days (for review, see Kumar et al., Curr Drug Targets 9:485-494, 2008). Allergen may be mixed with adjuvants prior to administration, for example with aluminium hydroxide or a mixture of aluminium and magnesium hydroxide, to promote a robust immune response. In an exemplary protocol, OVA-induced asthma model is generated in female Balb/c mice by dosing the animals at days 1 and 8 intraperitoneally with 200 μl of 50 μg/ml OVA in an adjuvant containing 40 mg/ml aluminium hydroxide and 40 mg/ml magnesium hydroxide (for example Imject Alum, Pierce, cat. No. 77176), followed by a 2 mg/ml OVA (in PBS) challenge intranasally at days 20, 21, 22, 23 and 24. An exemplary OVA-induced asthma model shows marked neutrophilia and elevated Th2 cytokine levels (IL-4, IL-5, IL-6) and marked eosinophilia in the lung homogenates beginning at days 21-25, and elevated serum OVA-specific IgE at day 15 post-OVA sensitization.

A house dust mite (HDM)-induced asthma model (Cates et al., J Immunol 173:6384-6392m 2004) is another exemplary model of allergic asthma. In this model, asthma is induced with HDM extracts from for example Dermatophagoides pteronyssinus or Dermatophagoides farina, allergens which are more representative of those that occur naturally when compared to the OVA challenge, and allows allergen sensitization via the respiratory mucosa. In an exemplary protocol, HDM-induced asthma model is generated in female Balb/c mice by intranasal administration of 25 ml of 2 mg/ml house dust mite extract on days 0-9. An exemplary HDM-induced asthma model shows marked neutrophilia, eosinophilia, increased lymphocyte and monocyte levels, elevated Th2 ctyokine leves (IL-4, IL-5), and increased mucus secretion in the lungs between days 1-11 post-HDM challenge. Airway hyperresponsiveness (AHR) is marked at day 11, and eosinophilia and increased lymphocyte and monocyte levels in the lungs persist throughout day 16 post-HDM challenge.

Other aeroallergens, such as fungal allergens (e.g. Aspergillus fumigates), pollen spores and ragweeds can also be used as an allergen to induce allergic asthma in an asthma model, and are reviewed in Fuchs and Braun, Curr Drug targets 9:495-502, 2008. In an exemplary protocol to generate fungus-induced asthma model, C57B16/J mice are sensitized intranasally with a preparation of soluble A. fumigates antigens, and at day 7 post-sensitisation are administered intratracheally with 5×10⁶ swollen conidia (or spores) suspended in PBS/Tween-80. A. fumigates models typically demonstrate persistent neutrophilia, eosinophilia, macrophage infiltration, airway hyperresponsiveness, airway remodeling such as subepithelial fibrosis and smooth muscle cell hyperplasia.

Allergens such as OVA or HDM can be administered chronically to reproduce chronic features of asthma such as airway remodeling and persistent AHR. Chronic challenge models are developed by increasing the number of allergen challenges (Lloyd, Curr Allergy Asthma Rep 7:231-236, 2007). Fungal spores persist in the lungs of sensitized mice thus obviating the need for repeated conidia challenges to develop chronic disease.

Preclinical asthma models reproducing features of asthma exacerbations are developed by infecting animals with for example rhinoviruses, such as respiratory syncytial virus (RSV) (Prince et al., Am J Pathol 93:771-791, 1978; Graham et al., J Med Virol 26:153-162 1988; Hegele et al., Eur Respir J 6:1324-1331, 1993). RSV can induce acute and chronic inflammation, acute bronchiolitis, AHR and airflow obstruction in animals. In an exemplary protocol anesthetized Balb/c mice are inoculated intranasally with 10⁷ PFU of RSV as described in Chavez-Bueno et al., Virology J 2:46-62, 2005.

Large animals such as pigs, sheep, primates and dogs with more “human-like” airway structure, morphology and function may be used as preclinical models of asthma and are reviewed in Van der Velden and Snibson, Pulm Pharmacol Ther Feb. 26, 2011 [Epub ahead of print].

One embodiment of the invention is a method of assessing efficacy of a therapeutic for the treatment of asthma, comprising:
a. providing the therapeutic;
b. providing a first and a second member of a preclinical model of asthma;
c. administering the therapeutic to the first member of the preclinical model of asthma and administering a vehicle to the second member of the preclinical model of asthma;
d. measuring the amount of pentraxin 3 in lung samples of the first member and the second member of the preclinical model of asthma;
e. determining that the lung sample of the first member of the preclinical model of asthma exhibits reduced amount of pentraxin 3 when compared to the amount of pentraxin 3 in the lung sample of the second member of the preclinical model of asthma.

The mode of administration of the therapeutics may be by any suitable route, for example parenteral administration, e.g., intradermal, intramuscular, intraperitoneal, intravenous, subcutaneous or intranasal. In vitro assays can be employed to help identify dosage ranges to be tested. Exemplary well known in vitro assays are inhibition of proinflammatory cytokine and chemokine production from cultured human bronchial epithelial cells, bronchial fibroblasts or airway smooth muscle cells.

The therapeutics can be administered at selected dosages in various carriers, such as a diluent, adjuvant, or excipient. Such carriers can be liquids, such as water and oils, including those of petroleum, animal, vegetable or synthetic origin, such as peanut oil, soybean oil, mineral oil, sesame oil and the like. For example, antibody and protein therapeutics may be administered in 0.4% saline and 0.3% glycine. Small molecules such as dexamethasone can be administered in water. Actual methods for preparing parenterally administrable compositions are well known and are described in more detail in, for example, “Remington's Pharmaceutical Science”, 15th ed., Mack Publishing Company, Easton, Pa.

PTX3 protein levels may be detected in any lung sample for example in lung homogenate, or in the supernatant of lung homogenates using well known methods, for example ELISA. Suitable PTX3 antibodies that can be used are commercially available and they can be tested for cross-reactivity across species. Additional PTX3 antibodies that are cross-reactive across species or specifically bind to PTX3 from one species can be made and be tested using well known methods (Ausubel et al., ed., Current Protocols in Molecular Biology, John Wiley & Sons, Inc., NY 1987-2001; Sambrook et al., Molecular Cloning: A Laboratory Manual, 2^{nd} Edition, Cold Spring Harbor, N.Y., 1989; Harlow and Lane, Antibodies, a Laboratory Manual, Cold Spring Harbor, N.Y., 1989; Colligan et al., ed., Current Protocols in Immunology, John Wiley & Sons, Inc., NY 1994-2001; Colligan et al., Current Protocols in Protein Science, John Wiley & Sons, NY, N.Y., 1997-2001; Kohler et al., Nature 256:495-497, 1975; U.S. Pat. No. 4,816,567, Queen et al., Proc. Natl. Acad. Sci. 86:10029-10033, 1989).

Lung homogenates are prepared by adding a whole lung to a Lysing Matrix D tube (MP Biomedicals) with 1 mL of PBS. The tube is homogenized in the Fast Prep Homogenizer for 45 seconds at a speed of 6.0. The tubes are then spun down in a centrifuge for 10 min at max speed. The supernant is collected and utilized for further assays. PTX3 levels are compared between lung samples of a member of a preclinical asthma model that has been administered a therapeutic and a member of a preclinical model of asthma that has been administered a control vehicle, such as phosphate buffered saline (PBS). Administration of a therapeutic to a preclinical model of asthma can reduce levels of PTX3 by at least 10%, for example by 10%, 15%, 20%, 25%, 30%, 35%, 40%, 45%, 50%, 55%, 60%, 65%, 70%, 75%, 80%, 85%, 90%, or 95% when compared to a preclinical model of asthma administered a control vehicle.

A therapeutic is assessed to be efficacious in the treatment of asthma when the therapeutic reduces the levels of PTX3 a significant degree (pvalue<0.05) in a sample derived from a preclinical model of asthma with administered therapeutic when compared to a corresponding sample from a preclinical model of asthma with administered control vehicle.

Any preclinical model of asthma can be used in the methods of the invention as long as PTX3 levels are upregulated in that preclinical model of asthma when compared to a suitable control, and are downregulated by known asthma treatments, such as glucocorticoids. PTX3 protein sequence for any species can be identified by routine cDNA cloning utilizing computer-based homology searches, PCR, or library screens if the protein sequence is now available.

The present invention will now be described with reference to the following specific, non-limiting examples.

### Example 1

### Pentraxin 3 is Upregulated in Preclinical Models of Asthma

### Materials and Methods:

### Ovalbumin (OVA)-Induced Asthma Model:

On Days 0, 7, and 14, female Balb/c mice (12 weeks old, Taconic, 6 animals per group) were dosed with OVA (Sigma, A-7641, stock is stored at 5 mg/mL) formulated in sterile PBS (100 μg/ml), and mixed on a rocker with an equal volume of Inject Alum (ready to use, Pierce, IL, cat no. 77161) at room temperature for more than 30 min. Final concentration of OVA was 50 μg/ml. The OVA/Alum was injected intraperitoneally 200 μl/mouse. On days 20-24, OVA was dissolved in sterile PBS to obtain a concentration of 2 mg/ml, and administered intranasally with 50 μl/mouse. In control mice, 50 μl/mouse of PBS was given intranasally. All mice were euthanized with an overdose of Nembutal and lung homogenates were collected immediately following euthanization. Mouse PTX3 ELISA (R&D Systems) was completed on lung homogenate. Cytospins were completed to obtain BAL differential counts using standardized methods. BAL Total cell counts were obtained using standardized methods. IL-4, IL-5, and IL-6 levels in the lung homogenate were obtained using the Mullipore Mouse 22-plex Luminex analysis kit.

### House Dust Mite (HDM)-Induced Asthma Model:

On days 0-9, female Balb/c mice (12 weeks old, Taconic, 6 animals per group) received 25 μl of high endotoxin HDM extract (2 mg/ml in phosphate buffered saline, Greer Labs, NC, cat no. XPB82D3A25, lot #129458) or 25 ml of sterile PBS vehicle control by intranasal administration (2 mg/ml of HDM reconstituted in PBS) and were anesthetized with isofluorane followed by intranasal administration of HDM extract (2 mg/ml) or PBS control, drop wise alternating between nostrils, in a total volume of 25 μl (50 μg HDM extract/day). All mice were euthanized with an overdose of Nembutal and lung homogenates were collected immediately following euthanization. Mouse PTX3 ELISA (R&D Systems) was completed on lung homogenate supernatant. Cytospins were completed to obtain BAL differential counts using standardized methods. BAL Total cell counts were obtained using standardized methods. IL-4 and IL-5 levels in the lung homogenate were obtained using the Mullipore Mouse 22-plex Luminex analysis kit.

A. fumigatus-Induced Asthma Model:

C57BL/6 mice were sensitized via intraperitoneal (i.p.) and subcutaneous (s.c.) route with a commercially available preparation of soluble A. fumigatus antigens dissolved in 0.2 ml of IFA. After three intranasal instillations of Aspergillus antigen, each spaced one-week apart, each mouse received 1×10⁷ swollen conidia suspended in 30 μl of PBS tween 80 (0.1%; vol/vol) via intratracheal (i.t.) administration. All mice were euthanized with an overdose of Nembutal and lung homogenates were collected immediately following euthanization. Mouse PTX3 ELISA was completed on lung homogenate supernatant.

The PTX3 levels were measured in lung homogenates from asthmatic mice by standard sandwich ELISA. Before ELISA, snap-frozen lung samples were thawed on ice and homogenized in a solution containing 2 mg of protease inhibitor (Complete; Boehringer Mannheim) per milliliter PBS containing 0.1% Triton X. PTX3 level was normalized to the total protein concentration in the tissue, as determined by the Bradford method (Bio-Rad Laboratories).

Bone marrow cells were flushed with RPMI 1640 (Mediatech, Herndon, Va.), from the long bones of näive wildtype mice. The bone marrow cells were then cultured with medium containing 20% FCS, 30% L-cell supernatant, L-glutamine, and penicillin/streptomycin (P/S) in RPMI 1640 for 6 days. The cells were centrifuged at 300×g for 10 min, the pellets were washed twice with PBS, and cells were incubated for overnight at 37° C. and 5% CO₂. During the M2 differentiation, cells were exposed to medium or M2 conditioning medium, containing recombinant mouse IL-4 (10 ng/ml), IL-13 (10 ng/ml) (all from R&D Systems, Minneapolis, Minn., USA), and incubated for 24 hours at 37° C.

### Results:

### OVA-Induced Asthma Model:

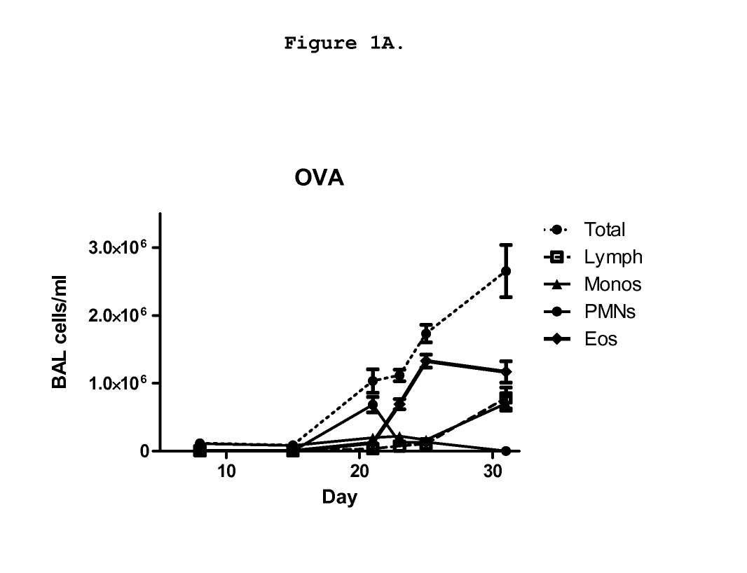

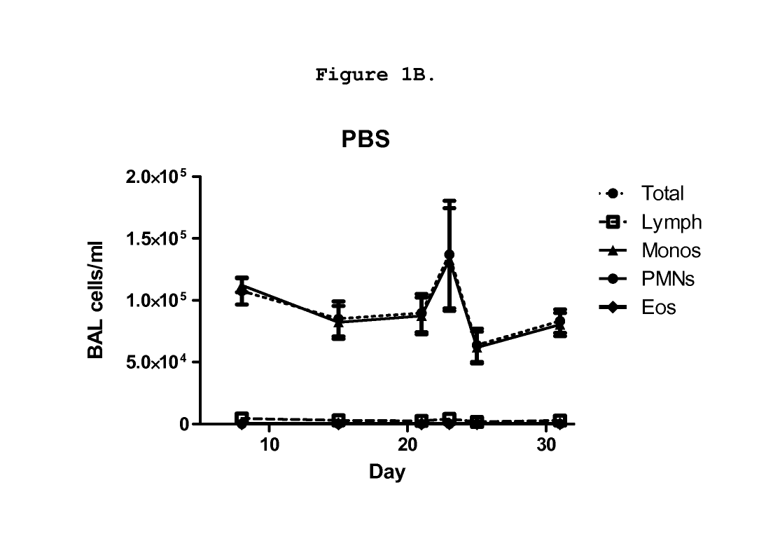

Marked neutrophilia, eosinophlila, increased monocyte and lymphocyte numbers were observed between days 21-31 in lung homogenates of OVA-induced asthma model when compared to control mice given PBS (FIGS. 1A and 1B, respectively).

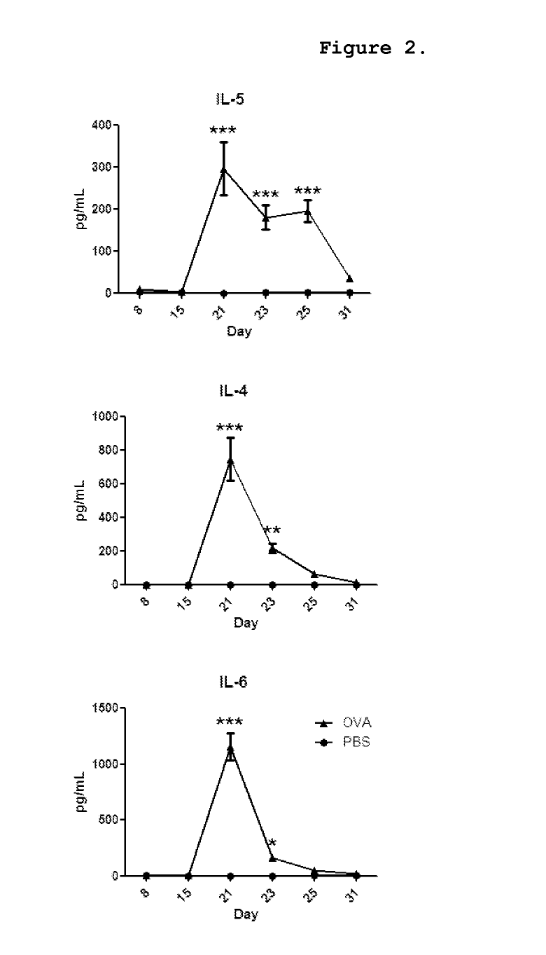

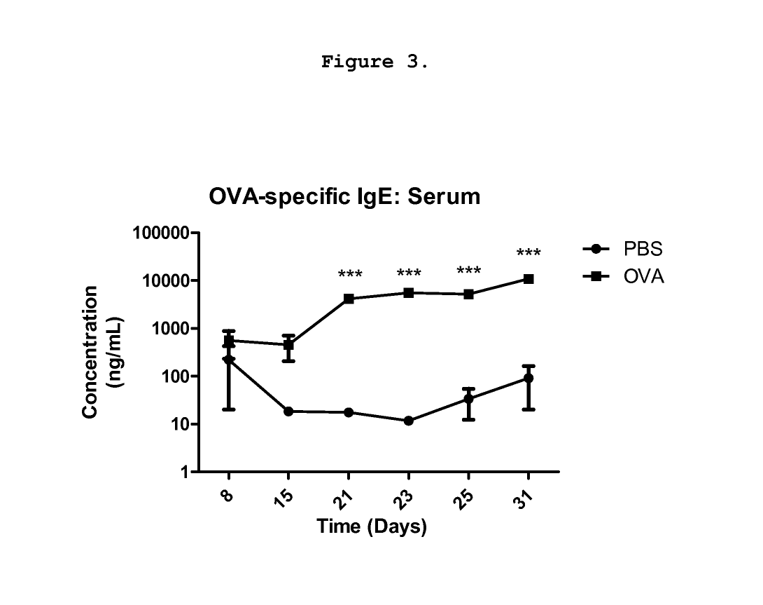

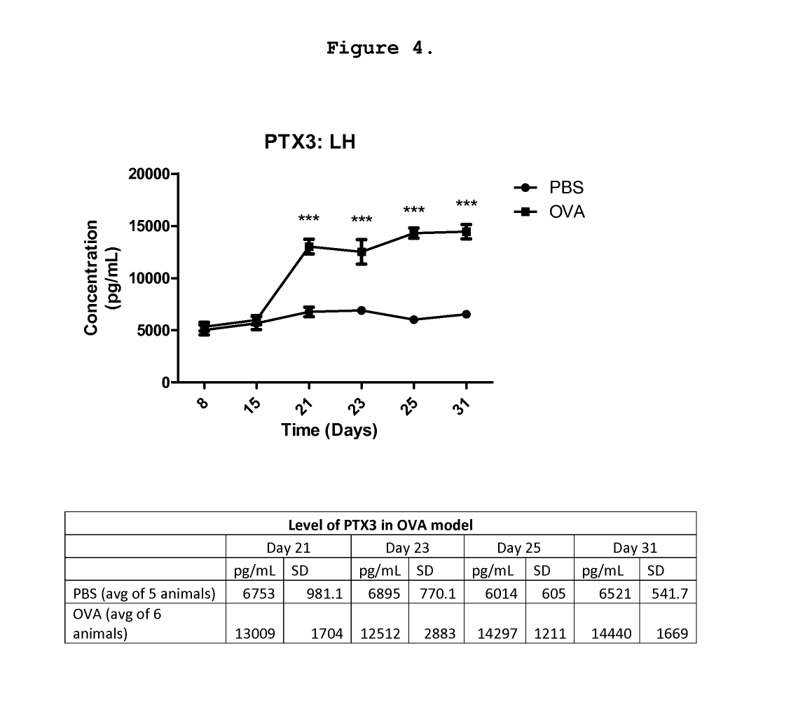

Th2 cytokines IL-4, IL-5 and IL-6 were increased in lung homogenates (FIG. 2) between days 21-25, depending on the cytokine. OVA-specific IgE was detected in serum after day 15 (FIG. 3). FIG. 4 shows the time course of protein levels of mouse PTX3 in lung homogenates following either OVA or PBS administration. After the intranasal challenge with OVA on day 21, significantly increased PTX3 levels were evident in the lung homogenates of OVA administered mice compared to the PBS administered mice (p<0.001). This statistically significant increase was present from day 21 to day 31 timepoints.

### HDM-Induced Asthma Model:

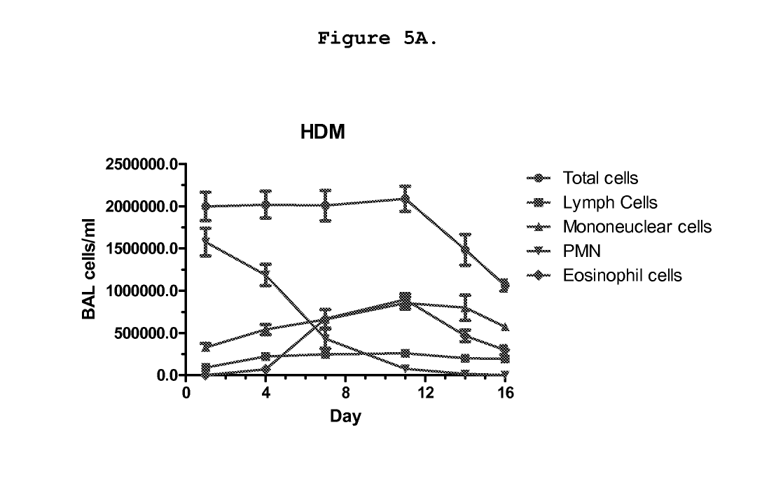

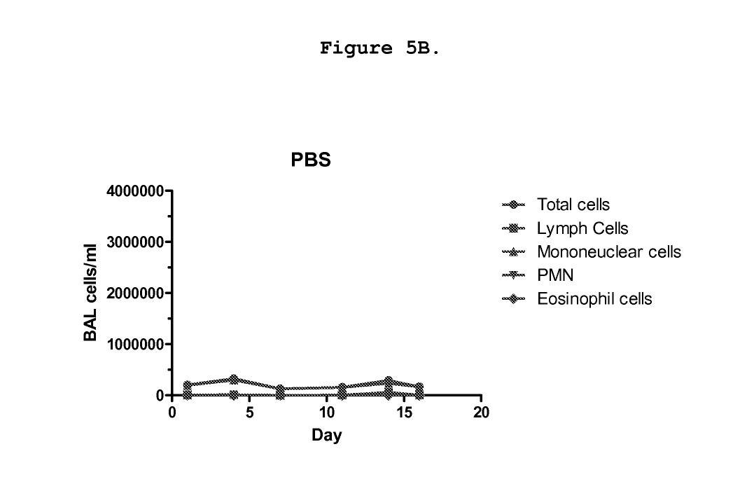

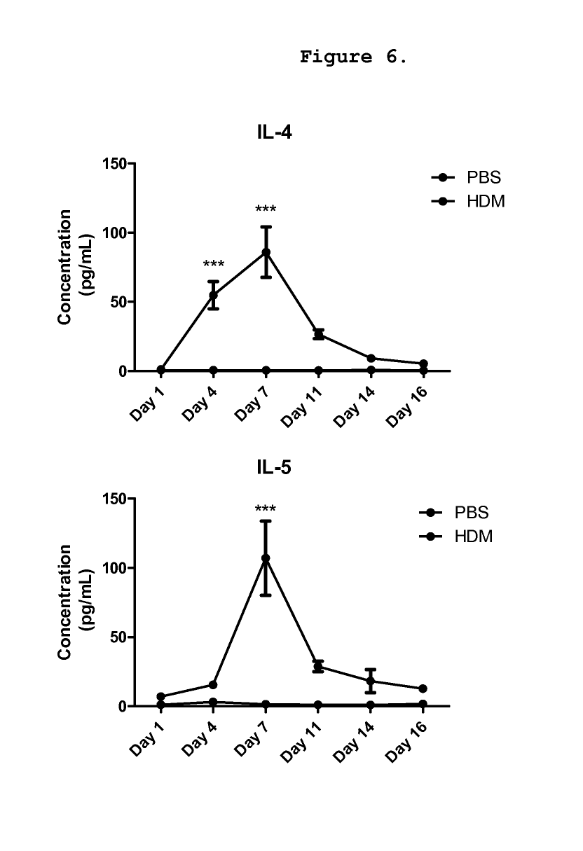

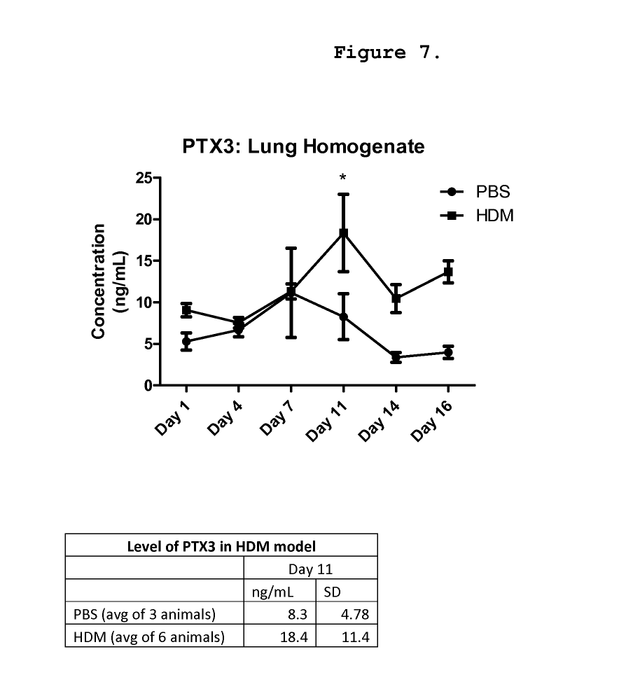

Neutrophilia, eosinophlila, and increased monocyte and lymphocyte numbers were observed between days 1-11 in lung homogenates of HDM-induced asthma model when compared to control mice given PBS (FIGS. 5A and 5B, respectively). Levels of IL-4 and IL-5 were elevated between days 1-11 in lung homogenates (FIG. 6). FIG. 7 shows the time course of protein levels of mouse PTX3 in lung homogenates following HDM intranasal administration. On Day 11, PTX3 levels were significantly increased in HDM administered mice compared to the PBS administered mice (p<0.05).

A. fumigates-Induced Asthma Model:

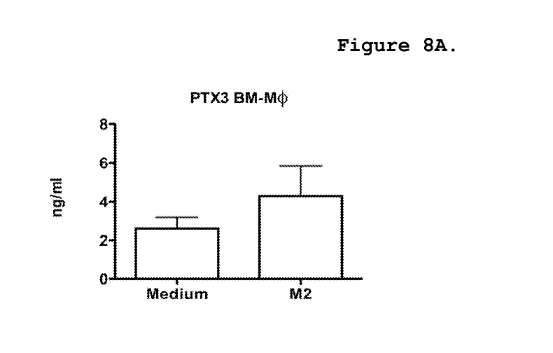

Increased levels of PTX3 were detected in bone marrow derived macrophages (M2) (FIG. 8A) and in lung homogenates (FIG. 8B) in A. fumigates-induced asthma model.

### Example 2

### PTX3 is Downregulated by Dexamethasone Treatment

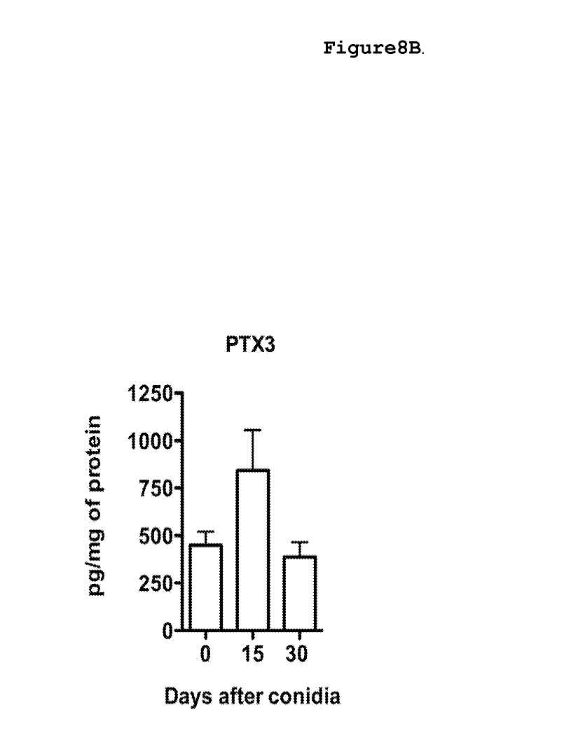

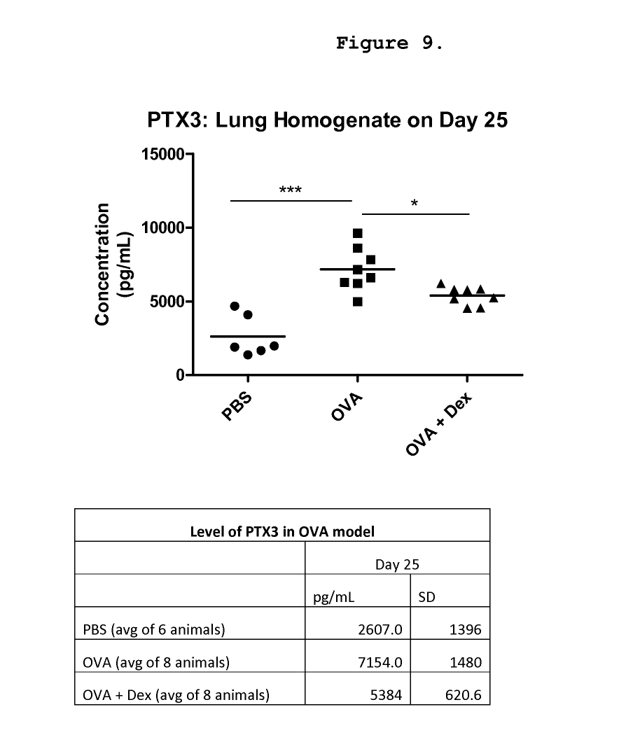

OVA-induced asthma model was generated as described above. Animals were dosed with dexamethasone at 1 mg/kg subcutaneously on days 21, 22, 23, and 24 given 2 hrs prior to the OVA challenge. Animals were sacrificed at day 25. PTX3 levels were reduced in the lung homogenates of animals given dexamethasone (FIG. 9).

### Example 3

### PTX3 is Upregulated in Human Lung Biopsy Samples Obtained from Asthmatic Subjects

12 (6 normal and 6 asthmatic) frozen lung samples (5-6 g total) were procured from Analytical Biological Services, DE. Lung homogenates were generated from 200 mg samples and placed with PBS and protease inhibitor cocktail in Fast Prep lysing matrix D tubes. Lung samples were homogenized for 45 seconds at a speed of 6.0 a single time. Tubes were then spun at 10,000 rpm for 5 min to achieve a clear supernatant. Human PTX3 ELISA (R&D Systems) was completed on lung homogenate supernatant and data was normalized to weight of tissue.

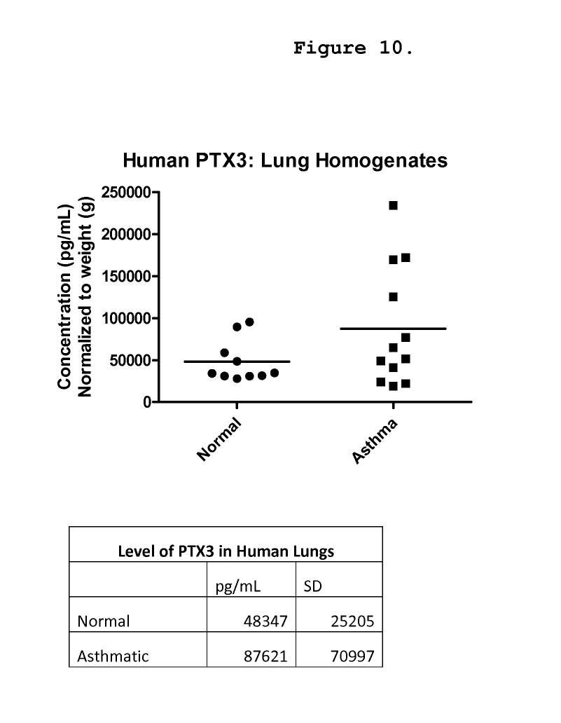

Protein levels of human PTX3 in lung homogenates of normal or asthmatic patients are shown in FIG. 10. There is a trend towards an increase in PTX3 in asthmatic lung homogenates when compared to normal lung homogenate samples.

### Example 4

### PTX3 Administration Enhances Inflammation During Allergic Airway Disease

### Materials and Methods:

A. fumigatus-Induced Asthma Model was Established as Described in Example 1.

Mice were treated every other day from day 0 to 15 or from day 15 to 30 with PBS or human PTX3 (R&D System, Minneapolis, Minn., USA; 10 μg/injection). At days 15 and 30 after conidia challenge, bronchial hyper-responsiveness was assessed in a Buxco™ plethysmograph (Buxco, Troy, N.Y.) after intravenous methacholine injection (420 μg/Kg). Other parameters were determined after the assessment of airway hyper-responsiveness.

### Results:

PTX3 involvement was studied in C57BL/6 mice that were sensitized and challenged with A. fumigatus antigens and live conidia (or spores). At days 15 after conidia the levels of PTX3 were increased 1.8-fold when compared with the control group, and at day 30 were similar to control (FIG. 8A). PTX3 was thus increased during the active inflammatory phase in this model

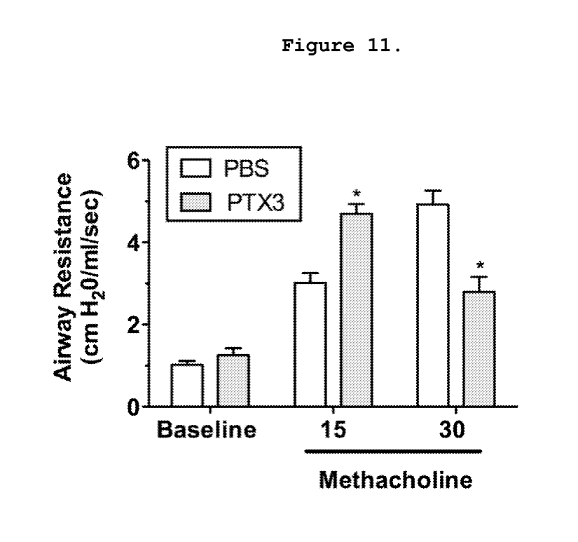

All groups of mice exhibited significantly increased airway resistance at days 15 and 30 after conidia when compared to the corresponding baseline resistance (FIG. 11). Exogenous PTX3 significantly increased the airway resistance in treated mice at day 15 after conidia. However, when administered from days 15 to 30 after conidia, exogenous PTX3 significantly decreased airway resistance at day 30 after conidia challenge.

### Exogenous PTX3 Exacerbates Cytokine Production in Asthma.

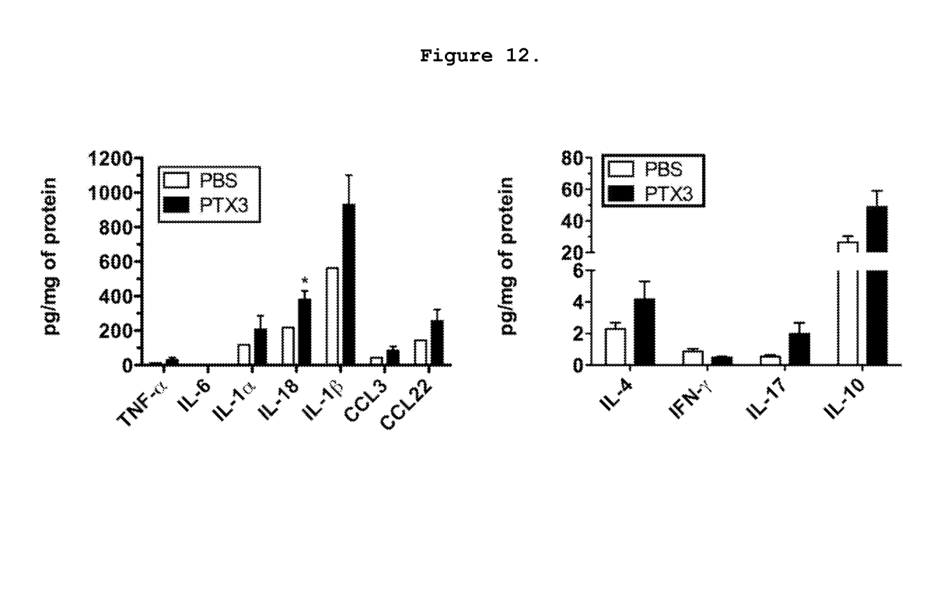

To investigate whether the treatment with exogenous PTX3 modified the cytokine profile in asthma, the levels of different cytokines in whole lungs homogenate from PBS and PTX3-treated mice at day 30 after conidia challenge were measured. PTX3 significantly increased IL-18 levels and induced a 1.7-fold increase in IL-1α, IL-1β, and CCL22 (MDC) levels when compared with the control group (FIG. 12). Also, PTX3 resulted in a 1.8-fold increase in IL-4 and IL-10, 3.7-fold increase in IL-17 and 1.8-fold reduction in IFN-γ compared with PBS treatment (FIG. 12).

Exogenous PTX3 increases airway inflammation, and induces the production of cytokines involved in the exacerbation of both inflammation and allergic response in fungal-induced airway disease. These results suggest that PTX3 could act as a potential biomarker for asthma disease.

The present invention now being fully described, it will be apparent to one of ordinary skill in the art that many changes and modifications can be made thereto without departing from the spirit or scope of the appended claims.

We claim:

1. A method of assessing efficacy of a therapeutic for the treatment of asthma, comprising:
a. providing the therapeutic;
b. providing a first and a second member of a preclinical model of asthma;
c. administering the therapeutic to the first member of the preclinical model of asthma and administering a vehicle to the second member of the preclinical model of asthma;
d. measuring the amount of pentraxin 3 in lung samples of the first member and the second member of the preclinical model of asthma;
e. determining that the lung sample of the first member of the preclinical model of asthma exhibits reduced amount of pentraxin 3 when compared to the amount of pentraxin 3 in the lung sample of the second member of the preclinical model of asthma.

2. The method of claim 1, wherein asthma is allergic asthma.

3. The method of claim 1, wherein asthma is characterized by eosinophilia, neutrophilia, airway hyperreactivity, or Th2 phenotype.

4. The method of claim 1, wherein the preclinical model of asthma is a house dust mite (HDM)-induced asthma model.

5. The method of claim 4, wherein the amount of pentraxin 3 is measured after at least 11 days from a first HDM challenge.

6. The method of claim 1, wherein the preclinical model of asthma is an ovalbumin (OVA)-induced asthma model.

7. The method of claim 6, wherein the amount of pentraxin 3 is measured after at least 21 days from a first OVA challenge.

8. The method of claim 6, wherein the amount of pentraxin 3 is measured after at least 25 days from the first OVA challenge.


SEQUENCE LISTING (5 sequences)

SEQ ID NO: 1 - protein, 381 aa, Homo sapiens
MHLLAILFCALWSAVLAENSDDYDLMYVNLDNEIDNGLHPTEDPTPCACGQEHSEWDKLFIMLENSQMRERMLLQATDDVLRGELQRLREELGRLAESLARPCAPGAPAEARLTSALDELLQATRDAGRRLARMEGAEAQRPEEAGRALAAVLEELRQTRADLHAVQGWAARSWLPAGCETAILFPMRSKKIFGSVHPVRPMRLESFSACIWVKATDVLNKTILFSYGTKRNPYEIQLYLSYQSIVFVVGGEENKLVAEAMVSLGRWTHLCGTWNSEEGLTSLWVNGELAATTVEMATGHIVPEGGILQIGQEKNGCCVGGGFDETLAFSGRLTGFNIWDSVLSNEEIRETGGAESCHIRGNIVGWGVTEIQPHGGAQYVS

SEQ ID NO: 2 - protein, 381 aa, Mus musculus
MHLPAILLCALWSAVVAETSDDYELMYVNLDNEIDNGLHPTEDPTPCDCRQEHSEWDKLFIMLENSQMREGMLLQATDDVLRGELQRLRAELGRLAGGMARPCAAGGPADARLVRALEPLLQESRDASLRLARLEDAEARRPEATVPGLGAVLEELRRTRADLSAVQSWVARHWLPAGCETAIFFPMRSKKIFGSVHPVRPMKLESFSTCIWVKATDVLNKTILFSYGTKWNPYEIQLYLSSQSLVLVVGGKENKLAADTVVSLGRWSHLCGTWSSEQGSMSLWANGELVATTVEMAKSHSVPEGGLLQIGQEKNGCCVGGGFDESLAFSGRITGFNIWDRVLSEEEIRASGGVESCHIRGNVVGWGVTEIQAHGGAQYVS

SEQ ID NO: 3 - protein, 381 aa, Rattus norvegicus
MHLPVILLCALWSAVLAETSYDYELMHVNLDNEIDNGLHPTEDPTPCDCRQEHSEWDKLFIMLENSQMREAMLLQATDDVLRGELQRLRAELGRLAGGMARPCSASGPADARLAQALEPLLQESRDANRRLARLEDAEARRHEATVPSLGAVLEELQRTRADLSAMQSWVARHWLPAGCETAIFFPMRSKKIFGSVHPVRPMKLESFSACIWVKATDVLNKTILFSYGTKWNPYEIQLYLSSQSLVFVVGGKENKLAADTVVSLGRWSHLCGTWSSEQGSMSLWANGELVATTLEMAKSHPVPEGGLLQIGQEKNGCCVGGGFDETLAFSGRITGFNIWDRVLSKEEIRASGGAESCHIRGNIVGWGVTEIQAHGGAQYVS

SEQ ID NO: 4 - protein, 381 aa, Pan troglodytes
MHLLAILFCALWSAVLAENSDDYDLMYVNLDNEIDNGLQPTEDPTPCDCGQEHSEWDKLFIMLENSQMREGMLLQATEDVLRGELQRLREELGRLAESLARPCAPGAPAEARLTSALDELLEATRDAGRRLARLEGAEAQRPEEAGRALAAVLEELRQTRADLHAVQGWAARSWLPAGCETAILFPMRSKKIFGSVHPVRPMRLESFSACIWVKATDVLNKTILFSYGTKRNPYEIQLYLSYQSIVFVVGGEENKLVAEAMVSLGRWTHLCGTWNSEEGLTSLWVNGELAATTVEMATGHIVPEGGILQIGQEKNGCCVGGGFDETLAFSGRLTGFNIWDSVLSNEEIRETRGAESCHIRGNIVGWGVTEIQPHGGAQYVS

SEQ ID NO: 5 - protein, 381 aa, Macaca mulatta
MHLLAILFCALWSAVLAENSDDYDLMYVNLDNEIDNGLHPTEDPTPCDCRQEHSEWDKLFTMLENSQMREGMLLQATDDVLRGELQRLREELGRLAESLARPCEQGALAEAKLASALDELLQASRDAGRRLERLEGAETQRPEEAGRALAAMLEELRQTRADLHAVQGWAARTWLPAGCDTAILFPMRSKKIFGSVHPVRPMRLESFSACIWVKATDVLNKTILFSYGTKRNPYEIQLYLSYQSIVFVVGGEENKLVAETMVSLGRWTHLCSTWNSEKGLTSLWVNGELVATTVEMATGHTVPEGGILQIGQEKNGCCVGGGFDETLAFSGRLTGFNIWDSVLSNEEIRETGGAESCHIRGNIVGWGVTEIQPHGGAQYVS